(19) United States
(12) Patent Application Publication
(10) Pub. No.: US 2023/0148949 A1
(43) Pub. Date: May 18, 2023

(54) Mechanism and Methods for Quantifying the Tactile Acuity of the Human Breast

(71) Applicant: THE UNIVERSITY OF CHICAGO, Chicago, IL (US)

(72) Inventors: Stacy Tessler Lindau, Chicago, IL (US); Sliman Bensmaia, Chicago, IL (US)

(21) Appl. No.: 17/987,649

(22) Filed: Nov. 15, 2022

Related U.S. Application Data
(60) Provisional application No. 63/280,807, filed on Nov. 18, 2021.

(51) Int. Cl. A61B 5/00

(52) U.S. Cl. CPC A61B 5/4312; A61B 5/0051; A61B 5/704; A61B 2562/0247

(57) ABSTRACT

Breast reconstruction and/or mastectomy may be performed to treat or prevent breast cancer or for a variety of other purposes. These procedures can result in disruption of the innervation of the breast, leading to loss of sensation. This loss of sensation can lead to diminishment of sexual function, sexual dysfunction, or other undesired effects. Devices and methods are provided for quantitatively assessing the tactile acuity of the breast at multiple locations. These embodiments can be applied to assess the natural acuity of the breast to design or calibrate an implanted system for restoring perception in the post-reconstruction breast. These embodiments can be also applied to assess the change in the perceptual acuity of the breast due to reconstruction or some other surgical intervention.

### CROSS-REFERENCE TO RELATED APPLICATION

The present application is a non-provisional patent application claiming priority to U.S. Provisional Patent Application No. 63/280,807, filed Nov. 18, 2021, the contents of which are hereby incorporated by reference.

### BACKGROUND

Breast reconstruction may be performed to restore breast aesthetics. Reconstruction may occur following mastectomy to treat or prevent breast cancer, following injury, or for some other reason. However, both mastectomy and breast reconstruction (and/or other surgical or radiation procedures performed on the breast or surrounding tissues) can result in diminishment or complete loss of sensory function or other functions (e.g., thermoregulation, lactation, mobility, etc.) of the breast. This loss or diminishment of sensory or other function can have a variety of negative psychological, physiological, social, and/or relational effects, including sexual dysfunction and/or diminishment or loss of sexual function

### SUMMARY

In a first aspect, a system is provided that includes: (i) a padded platform, wherein the padded platform includes an aperture, and wherein the padded platform is shaped to permit a patient to lay prone on the platform with a breast of the patient located within the aperture; (ii) a sensor configured to detect at least one of a pressure, a force, or a displacement at a sensor probe of the sensor; (iii) an armature, wherein the sensor is coupled to the armature, and wherein the armature is configured to bring the sensor probe into contact with the breast of the patient when the patient is laying prone on the platform such that the breast of the patient is located within the aperture; (iv) a user interface; and (v) a controller. The controller is operably coupled to the sensor and the user interface and is configured to: (a) operate the sensor during a first period of time to detect at least one of a pressure, a strain, a force, or a displacement at the sensor probe when the sensor probe is brought into contact with the breast of the patient; and (b) operate the user interface to receive a first input during or after the first period of time that is indicative of whether the patient experienced a specified percept during the first period of time.

In a second aspect, a system is provided that includes: (i) a padded platform, wherein the padded platform includes an aperture, and wherein the padded platform is shaped to permit a patient to lay prone on the platform with at least one of: a breast of the patient located within the aperture, a portion of a chest of the patient spanning the aperture, or a portion of an abdomen of the patient spanning the aperture; (ii) a sensor configured to detect at least one of a pressure, a force, or a displacement at a sensor probe of the sensor; (iii) an armature, wherein the sensor is coupled to the armature, and wherein the armature is configured to bring the sensor probe into contact with skin of the breast, chest, or abdomen of the patient when the patient is laying prone on the platform such that at least one of: the breast of the patient is located within the aperture, the portion of the chest of the patient is spanning the aperture, or the portion of the abdomen of the patient is spanning the aperture; (iv) a user interface; and (v) a controller. The controller is operably coupled to the sensor and the user interface and is configured to: (a) operate the sensor during a first period of time to detect at least one of a pressure, a strain, a force, or a displacement at the sensor probe when the sensor probe is brought into contact with the skin of the patient; and (b) operate the user interface to receive a first input during or after the first period of time that is indicative of whether the patient experienced a specified percept during the first period of time.

In a third aspect, a method for quantitatively assessing tactile acuity of a breast of a patient is provided that includes: (i) bringing a sensor probe of a sensor into contact with a breast of a patient when the patient is laying prone on a padded platform such that the breast of the patient is located within an aperture of the padded platform; (ii) operating a sensor during a first period of time to detect at least one of a pressure, a strain, a force, or a displacement at a sensor probe of the sensor when the sensor probe is in contact with the breast of the patient; and (iii) operating a user interface to receive a first input during or after the first period of time that is indicative of whether the patient experienced a specified percept during the first period of time.

### BRIEF DESCRIPTION OF THE DRAWINGS

The patent or application file contains at least one drawing executed in color. Copies of this patent or patent application publication with color drawings will be provided by the Office upon request and payment of the necessary fee.

The accompanying drawings are included to provide a further understanding of the system and methods of the disclosure and are incorporated in and constitute a part of this specification. The drawings illustrate one or more embodiment(s) of the disclosure, and together with the description serve to explain the principles and operation of the disclosure.

FIG. 1A depicts aspects of an example system for assessing the tactile acuity of the breast(s), chest, and/or abdomen of a patient.

FIG. 1B depicts aspects of an example system for assessing the tactile acuity of the breast(s), chest, and/or abdomen of a patient.

FIG. 2 depicts experimental results comparing breast sensitivity to hand sensitivity. Breast sensitivity was measured using systems as described herein.

FIG. 3 depicts a schematic of the medial and lateral areas of breast, hand, and back as assessed in tactile acuity measurements, a set of tactile acuity scales used to perform such measurements (distances in mm), and the measured just-noticeable differences (JND) in tactile acuity for hand, back, lateral breast, and medial breast.

FIG. 4 depicts an experimentally measured relationship between bust size and

spatial acuity.

FIG. 5 depicts aspects of an example system.

FIG. 6 depicts aspects of an example method.

### DETAILED DESCRIPTION

The following detailed description describes various features and functions of the disclosed systems and methods with reference to the accompanying figures. The illustrative system and method embodiments described herein are not meant to be limiting. It may be readily understood that certain aspects of the disclosed systems and methods can be arranged and combined in a wide variety of different configurations, all of which are contemplated herein.

### I. Overview

Breast reconstruction or other surgical or radiation interventions to the breast or surrounding tissues (e.g., the intercostal nerves that innervate the breast) or certain anatomic conditions or injuries can result in diminished or lost sensation of the breast, reduced thermoregulation or temperature perception, lost or diminished lactation, lost or diminished mobility, or other reductions in the various natural functions of the breast. While the surgical techniques or approaches employed to obtain a particular surgical, radiation, or other treatment goal can be adapted to spare the relevant nervous tissue, such adaptation may not be available in every case. Further, such adaptations may not always be successful, resulting in diminished or lost sensory or other aspects of breast function. Such surgical or other therapeutic interventions can include mastectomy, radiation, or other procedures to remove or otherwise diminish cancerous tissue in the breast or surrounding tissue, gender reassignment procedures, reconstruction following injury, elective cosmetic procedures, or other procedures that may result in loss or diminishment of breast function.

It can be beneficial in such circumstances to quantitatively assess the sensory acuity of the breast before and after such interventions. Such assessment could be done to assess the effects of such interventions on the sexual and/or sensory function of the breast. Such information could be used to plan or select the intervention, e.g., to select an intervention location, approach, or type to maximally spare the observed pattern of sensory acuity in a particular patient's breast. Additionally or alternatively, such assessments could be performed on a population of patients to determine the expected sensory effects of different interventions at the population level. Such information could be used to provide information on the relative merits of the different interventions in the absence of patient-specific acuity data, allowing a patient to make an informed decision about their care with respect to a variety outcome factors including the probable effects on breast sensory function. Such quantitative data about the sensory function of the breast could also be used to design and/or calibrate a prosthetic system for restoring breast sensory function following reconstruction or other surgical interventions.

For example, to restore breast function lost due to surgical intervention or other medical treatment or trauma, systems (e.g., worn or implanted devices, materials, monitors, information systems, or some combination thereof) may be provided. In particular, breast sensory and other functions may be restored by implanting or otherwise placing one or more sensors in or on the breast to detect physical variables such as movement, vibration, pressure (e.g., deep, superficial, fine, and/or gross pressure), wetness, suction, air movement, temperature, strain, deformation, accelerations, variables related to psychosocial cues, or other physical variables related to a lost sensation (e.g., proprioception, touch, texture, deep or superficial pressure, erection of the nipple (e.g., in response to physiological arousal), tingling, fullness, heat, or other sensations related to “letting down” in relation to nursing (e.g., when thinking about nursing, when hearing a baby cry)). These detected physical variables can then be used to determine a stimulus that is provided to the central nervous system (e.g., via electrical stimulation of one or more peripheral nerves) such that a percept physiologically relevant to the detected physical variables is experienced. The size, sensor resolution, sensor dynamic range, mapping of sensor outputs to electrical stimuli, or other properties of such a system could be determined based on breast sensory acuity data generated using the systems or methods described herein. This could include one (or a small number) of such sensory prosthetic systems being designed with a sensor size, resolution, dynamic range, or other properties set based on sensory acuity data assessed from a population of patients. Additionally or alternatively, sensory acuity data assessed from an individual patient prior to a surgical intervention could be used to calibrate, program, size, or otherwise customize a sensory prosthetic system used by (e.g., implanted in) the individual patient.

It can also be beneficial to quantitatively measure such sensory acuity data for the breast, chest, and/or abdomen for other purposes. For example, to perform basic research, to investigate the variation in skin innervation between people, to assess sensory acuity as a diagnostic for some other disease or condition (e.g., multiple sclerosis, trauma, lupus, Guillain-Barre syndrome, diabetes, cancer), to evaluate hormonal changes and/or effects on sexual or non-sexual sensory function, to evaluate healing after a surgery or other trauma, or to provide information for some other application.

However, in practice it is difficult to perform quantitative sensory acuity assessments (e.g., a two-point discrimination test, a test of vibrotactile sensitivity at one or more frequencies of stimulation) on the breast, chest, and/or abdomen. This is because a patient to be tested in such a manner is breathing, which can affect the test due to the constant breathing-related changes in the location of the skin of the breast, chest, and/or abdomen. In contrast, performing such tests on the hands, feet, arms, legs, etc. can be simplified by securing the body part of interest (e.g., using straps or other means) or even just by the patient holding the boy par to interest still against a platform or other object and then performing the test against the unmoving body part.

The systems and methods described herein provide for improved quantitative sensory acuity assessment of the breast(s), chest, and/or abdomen even in the presence of breathing by providing securing platforms or other elements to control the location of the breast, chest, and/or abdomen (and/or skin therefor) while allowing breathing to continue unimpeded. These systems and methods include providing a padded platform on which a patient can lay. The padded platform includes at least one aperture through which sensory acuity tests can be performed on skin of body parts of the patient that are located within the aperture (e.g., skin of the breast(s) and/or portions of the chest of the patient) and/or that span across the aperture (e.g., skin of portions of the chest and/or abdomen of the patient). The patient's breathing is unimpeded by their laying on the platform; that breathing results in motion of their back or other body parts away from the platform. However, the location of skin of the breast(s), chest, and/or abdomen of the patient, that is accessible through the aperture, is now relatively unaffected by breathing motion, and so sensory acuity tests can be applied thereto. Such systems and methods can be further improved by also using a plate to contact the skin of interest (e.g., skin of the breast, chest, or abdomen that it located within the aperture and/or that span across the aperture). Such a plate can further secure the location of the skin against breathing-related motion, and hole(s) formed through the plate can allow access to the skin for the application of sensory acuity tests.

The methods described herein for quantitatively assessing sensory acuity (e.g., tactile sensory acuity) of the breast, chest, and/or abdomen include providing a percept to the breast, chest, and/or abdomen of a patient and measuring a physiological effect/reaction of the provided percept in the patient and/or receiving feedback from the patient regarding the percept (e.g., whether the patient perceived the percept, whether the percept increased, decreased, moved, or otherwise differed from a previously-applied percept). A property of the applied percept (e.g., a pressure, force, displacement, location, etc. in one or more dimensions) can be detected to measure one or more physical attributes of the applied percept. To provide comfort to the patient while facilitating repeatability and the ability to objectively measure one or more properties of the applied percepts, a platform (e.g., a padded massage table) is provided on which the patient can lay in a prone position (i.e., downward-facing). The platform includes an aperture into which the patient's breast(s) can be placed and/or across which the patient's chest or abdomen can span, permitting a measurement apparatus to access the breast(s), chest, and/or abdomen, and thus to provide percepts thereto and detect physiological effects/reactions (e.g., vasocongestion of breast, nipple erection, arousal, etc.), from below through the aperture.

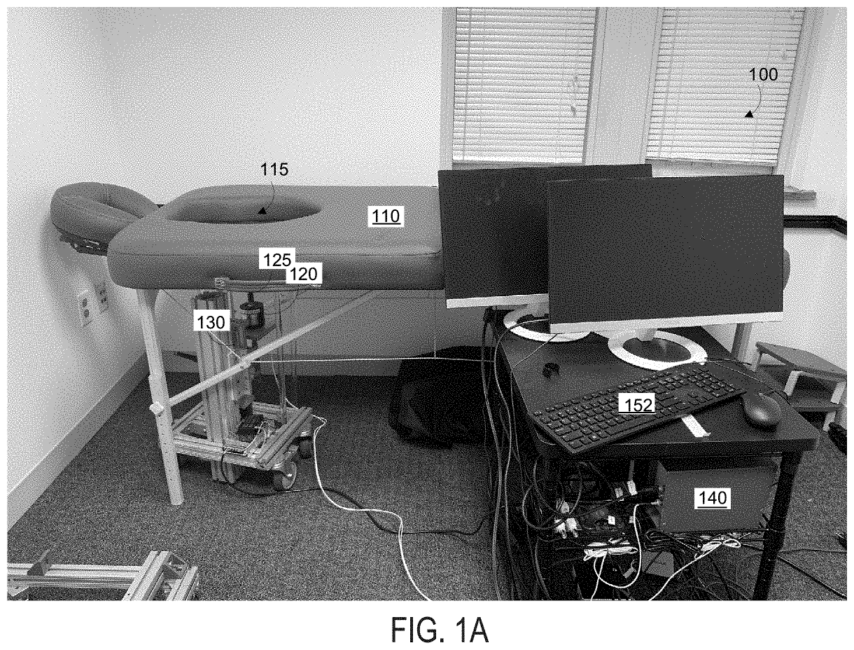

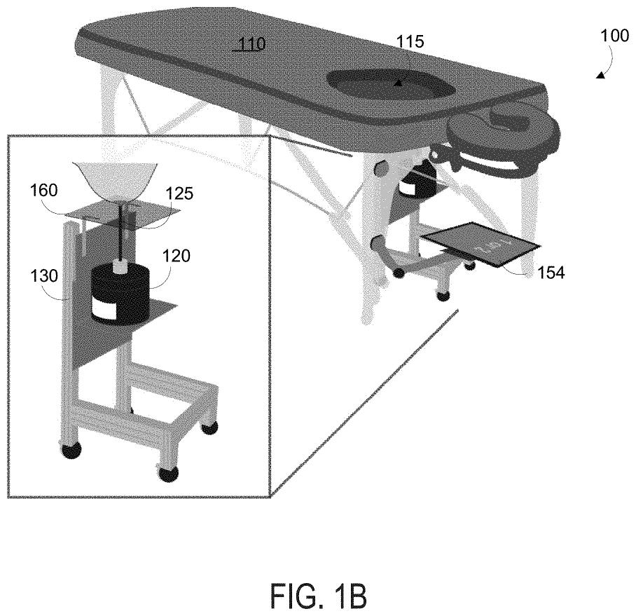

FIGS. 1A and 1B depict an example of a system 100 as described herein for assessing the tactile acuity of the breast(s), chest, and/or abdomen of a patient. The system 100 includes a padded platform 110 that has an aperture 115. A patient can lay prone on the padded platform 110 such that their breasts are located within aperture 115 and thus accessible from below to provide percepts to assess the tactile acuity of the breast(s). A sensor 120 is coupled to a sensor probe 125. The sensor probe 125 is configured to contact the patient's breast to provide percepts thereto. The sensor 120 is coupled to the sensor probe 120 such that the sensor 120 can operate to detect a pressure, force, displacement, or other properties of percepts applied to the breast via the sensor probe 125. The system 100 also includes an armature 130 that can be used to adjust the location of the sensor probe 125 relative to the breast of a patient, thereby allowing the location, pressure, force, or other properties of applied percepts to the modified and/or controlled. In some examples, the system could include an actuator (e.g., a servo, motor, a hydraulic or pneumatic cylinder) coupled to the armature 130, thereby facilitating automated application of percepts. Additionally or alternatively, the system 100 could include levels, wheels, or other elements to facilitate manual application or control of percepts to the breast.

As shown, the aperture 115 is a single static aperture formed through the padded platform 110 that is sized and positioned to permit one or both breasts of a patient to be located within the aperture 115 and accessible by the sensor probe 125 when the patient is laying prone on the padded platform 110. However, this is only one possible configuration of such an aperture and/or padded platform. For example, the size and/or location of the aperture within the padded platform could be changed to permit access to a single breast of a patient and/or to some other portion of the chest and/or abdomen of a patient for assessment of tactile sensory acuity of skin thereof. In some examples, this could include the padded platform having formed therethrough multiple apertures, with padded plugs being insertable therein (e.g., by being mechanically bolted, latched, or otherwise secured to a frame of the padded platform) to allow a single one of the apertures to be selected for use as described herein (e.g., by removing the plug from only the selected aperture(s)).

The system 100 also includes a controller 140 (e.g., desktop computer, a server, a laptop computer, a tablet computer) coupled to the sensor 120 and other elements of the system 100 (e.g., a display, keyboard, button, or other user interface elements, an actuator configured to facilitate automated generation of percepts). The system additionally includes a user interface for receiving inputs indicative of whether a patient perceived an applied percept, perceived a chance in an applied percepts, a degree or direction of change in an applied percept, or some other property of one or more percepts applied to the breast of the patient via the sensor probe 125. Such a user interface could include a keyboard 152 (e.g., for a clinician to input feedback provided verbally by a patient), one or more buttons (not shown) which could be usable by a patient when laying on the padded platform 110 to provide feedback related to applied percepts, or other elements for inputting data into the system 100. The user interface can also include output elements, e.g., a display 154 that a patient could observe when laying on the padded platform 110 and which could be used to provide instructions or other indications to the patient to facilitate the patient providing useful feedback related to percepts applied to the patient's breast(s).

The sensor(s) 120 of the device may include a variety of elements configured to detect stress, strain, vibration, pressure, deformation, motion (e.g., relative or absolute velocity and/or acceleration), orientation (e.g., relative to the padded platform 110, to the patient's nipple or breast), relative location (e.g., relative location between elements of the system portions of the patient's nipple or breast and/or previously locations of the elements of the system when providing prior percepts), temperature, vasodilation, or some other physical variable(s) related to the breast and/or to sensations that might be experienced by the patient. Cameras, encoders, or other additional sensors could be included to, e.g., detect deformation of the breast due to movement of the sensor probe, to detect the location of the sensor probe 125 relative to the breast and/or tissues thereof (e.g., the nipple, the nipple areolar complex) and/or relative to prior locations of the sensor probe 125 (e.g., when providing previous percepts to the breast), or to detect some other variables of interest (e.g., one or more physiological responses) in assessing the sensory acuity of the breast.

The system 100 could include additional outputs for providing percepts to the breast. For example, the system could include one or more vibrators, heaters, or other stimulators for providing controlled vibration, heat, or other percepts to the breast. Properties of such percepts could be detected, confirmed, and/or controlled via feedback detected using the sensor 120 and/or additional sensors (e.g., additional sensor(s) located in the sensor probe 125). In some examples, an actuator (not shown) that is used to control the location of the sensor probe 125 (e.g., to place the sensor probe 125 in contact with a specified potion of a patient's skin, to move the sensor probe to contact a different portion of the patient's skin) could also be used as a stimulator to provide stimuli to the skin, e.g., to perform an indentation test.

A variety of different stimuli could be provided by a system as described herein. For example, a specified force and/or displacement could be applied to the skin by the sensor probe 125 to assess pressure sensitivity, two-point discrimination, sensitivity to displacement, sensitivity to lateral strain, or sensitivity to some other physical property at a location on the patient's skin could be assessed. Where multiple sensor probes are provided and/or wherein the sensor probe 125 is bifurcated, two-point discrimination or sensitivity to some other physical property at two or more locations on the patients' skin could be assessed. Where the sensor probe 125 includes or is coupled to a heater and/or a cooling elements, sensitivity to temperature and/or heating of the patient's skin could be assessed. Vibration at one or more specified magnitudes and frequencies could be applied, via the sensor probe 125, to assess vibrotactile sensitivity of the skin. Such vibration could be provided by a vibration exciter capable of generating vibrations across a range of physiologically relevant frequencies, e.g., from 50 to 1000 Hz. As different nerve fibers transmit vibrosensory information at different frequencies, such a vibration exciter-type stimulator could be operated to provide stimulation to skin at those different frequencies to measure differences in the cutaneous innervation, vibrotactile acuity, or other properties of vibrotactile sensation at the different frequencies. For example, a first low frequency stimulus could be provided to skin at around 10 Hz (e.g., between 5 Hz and 30 Hz) and a second high-frequency stimulus could be provided to skin at higher frequencies around 250 Hz (e.g., between 200 Hz and 300 Hz).

The magnitude, frequency, or other properties of applied stimuli could be determined based on the commands, voltages, currents, etc. used to control the stimulator or other element(s) used to generate the stimuli. Additionally or alternatively, a sensor (e.g., sensor 120) could be used to independently measure and/or verify the properties of the applied stimulus. For example, an accelerometer, gyroscope, pressure sensor, temperature sensor, or other type of sensor could be disposed in the sensor probe 125 (e.g., proximate to the point of contact with skin) and used to measure a magnitude or frequency of an applied vibratory stimulus, a temperature or heat flux of an applied thermal stimulus, a force, pressure, or displacement of an applied indentation stimulus, a force, pressure, or displacement of an applied lateral force stimulus, or some other properties of an applied stimulus. Such operation could be done to improve the accuracy of sensory acuity measurements made using the systems and methods described herein (e.g., as compared to relying on the calibration, accuracy, resolution, or other properties of a stimulator used to generate the sensory stimuli).

The controller 140 could operate to provide a variety of different percepts (e.g., different levels of force to different locations of the breast) and to receive inputs indicative of a patient's perception thereof. Such information could then be used to quantitatively assess the sensory acuity of a patient's breast. This could include determining pressure sensitivity, two-point discrimination, sensitivity to vibration at one or more frequencies, sensitivity to displacement, sensitivity to lateral strain, sensitivity to heat, or sensitivity to some other physical property at one or more locations of the breast. Where the system 100 includes an actuator, the application of a series of percepts to the breast could be accomplished in an automated or semi-automated manner. Alternatively, such different percepts could be manually applied/modified (e.g., according to a program presented to a clinician via a display) and their quantitative properties (e.g., pressure, force, displacement) detected using the sensor 120.

The interface between the sensor probe 125 (or some other means for delivering stimuli to skin) and the skin can be further stabilized by the system 100 including a plate 160. The plate 160 is configured to be put into contact with skin (e.g., skin of the patient's breast) in order to provide a controlled contact therewith. The plate 160 also include a hole formed therethrough, via which the sensor probe 125 (or other stimulation means) can access the skin, through the plate, to provide stimuli. The stabilization of the skin-probe contact provided by such a plate 160 can also facilitate more highly-calibrated tests. Such a plate could allow the degree of indentation of the skin for a test to be more accurately and consistently controlled and/or measured. For example, a vibrotactile assessment could be performed by indenting the skin by a specified amount (e.g., 0.5 mm) before providing the vibratory stimulus, in order to control for the effects of indentation depth. The presence of the plate (and the hole formed therein) could allow such specified-depth indentations to be made more consistently and/or with less effort (e.g., by operating an actuator to automatically move the end of the sensor probe 125 to a location 0.5 mm beyond the upper plane of the plate 160).

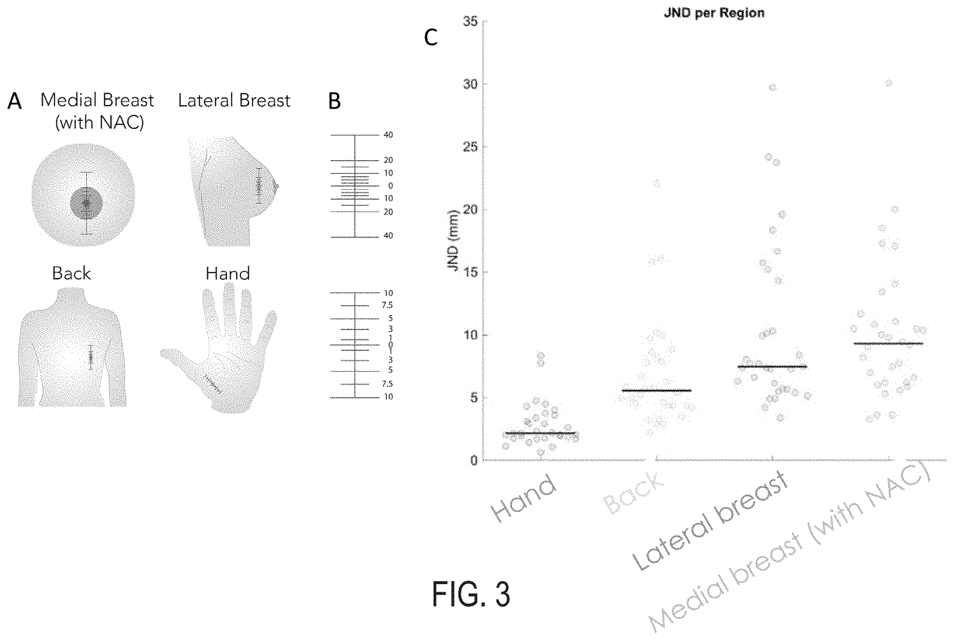

The plate 160 could have a single hole, or multiple. Multiple holes could be provided in the plate 160 to facilitate assessment of sensory acuity at multiple locations across the skin without moving the plate. Accordingly, the spacing and pattern of holes in the plate could be specified to correspond to a pattern of locations of sensory stimuli provided as part of a battery of stimuli, e.g., to correspond to the pattern of stimulus locations applied in a two-point discrimination test (e.g., the pattern(s) shown in pane “B” of FIG. 3). Additionally or alternatively, such multiple holes through the plate 160 could be used to provide stimuli to multiple locations simultaneously and/or at shorter time differences than would be possible by moving a single sensor probe 125 (or other stimulation means) from one stimulus location to another. For example, two or more holes through the plate 160 could be used to measure two-point discrimination.

### II. Experimental Results

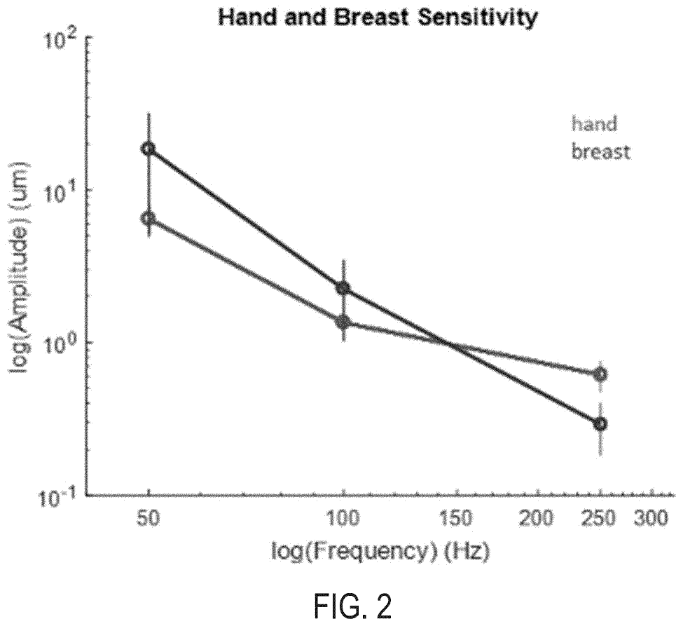

In a first experiment, breast sensitivity was measured and compared to hand sensitivity. Results are shown in FIG. 2.

In a second experiment, breast tactile acuity was compared to hand and back tactile acuity. Results are shown in FIG. 3. Pane ‘A’ of FIG. 3 depicts a schematic diagram of the regions tested and the manner of the testing. Pane ‘B’ of FIG. 3 depicts schematic diagrams of the spacing of the stimuli presented in the experiments, in millimeters. Pane ‘C’ of FIG. 3 depicts the experimental results measured for just noticeable difference (JND) for each evaluated region.

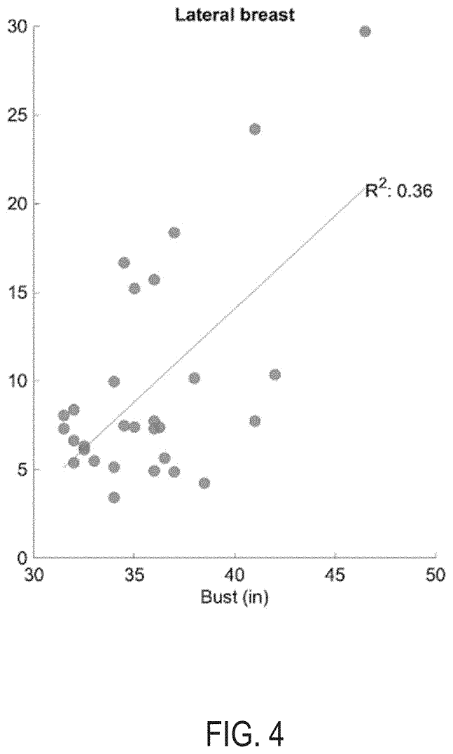

In a third experiment, breast tactile acuity was measured as a function of bust size (measured in inches). Results are shown in FIG. 4.

### III. Example Systems

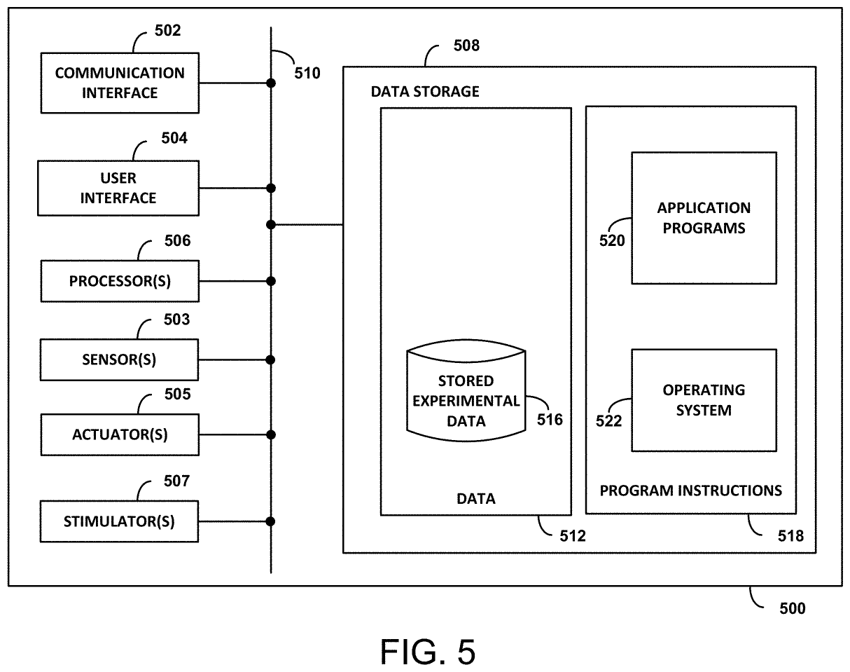

FIG. 5 illustrates an example system 500 that may be used to implement the methods described herein. By way of example and without limitation, system 500 may be or include a computer (such as a desktop, notebook, tablet, or handheld computer, a server), elements of a cloud computing system, or some other type of device or system. It should be understood that elements of system 500 may represent a physical instrument and/or computing device such as a server, a particular physical hardware platform on which applications operate in software, or other combinations of hardware and software that are configured to carry out functions as described herein.

As shown in FIG. 5, system 500 may include a communication interface 502, one or more sensors 503, a user interface 504, one or more actuators 505, one or more processors 506, one or more stimulators 507, and data storage 508, all of which may be communicatively linked together by a system bus, network, or other connection mechanism 410.

Communication interface 502 may function to allow system 500 to communicate, using analog or digital modulation of electric, magnetic, electromagnetic, optical, or other signals, with other devices (e.g., with databases that contain sets of training inputs or related data, e.g., map data that can be updated based on additional user inputs), access networks, and/or transport networks. Thus, communication interface 502 may facilitate circuit-switched and/or packet-switched communication, such as plain old telephone service (POTS) communication and/or Internet protocol (IP) or other packetized communication. For instance, communication interface 502 may include a chipset and antenna arranged for wireless communication with a radio access network or an access point. Also, communication interface 502 may take the form of or include a wireline interface, such as an Ethernet, Universal Serial Bus (USB), or High-Definition Multimedia Interface (HDMI) port. Communication interface 502 may also take the form of or include a wireless interface, such as a WiFi, BLUETOOTH®, global positioning system (GPS), or wide-area wireless interface (e.g., WiMAX, 3GPP Long-Term Evolution (LTE), or 3GPP 5G). However, other forms of physical layer interfaces and other types of standard or proprietary communication protocols may be used over communication interface 502. Furthermore, communication interface 502 may comprise multiple physical communication interfaces (e.g., a WiFi interface, a BLUETOOTH® interface, and a wide-area wireless interface).

In some examples, communication interface 502 could be used to communicate with sensors (e.g., of sensors 503), actuators (e.g., of actuators 505), and/or stimulators (e.g., of stimulators 507) that are physically separate from the system 500 (e.g., that are part of a separate system, which may include its own processors, communications interface, etc.) to allow the system 500 to control such separate elements as part of an experimental protocol. For example, a sensor that is battery-powered and that includes a wireless communication functionality could be operated by the system 500 using the communication interface 502 and/or the system 500 could receive sensor data form such a sensor using the communication interface 502.

User interface 504 may function to allow system 500 to interact with a user, for example to receive input from and/or to provide output to the user. Thus, user interface 504 may include input components such as a keypad, keyboard, touch-sensitive or presence-sensitive panel, computer mouse, trackball, joystick, microphone, and so on. User interface 504 may also include one or more output components such as a display screen which, for example, may be combined with a presence-sensitive panel. The display screen may be based on CRT, LCD, and/or LED technologies, or other technologies now known or later developed. User interface 504 may also be configured to generate audible output(s), via a speaker, speaker jack, audio output port, audio output device, earphones, and/or other similar devices. The user interface 504 may be operable to permit a user to setup and run experimental protocols to assess the tactile acuity or other tactile properties of the skin of one or more patients at one or more locations on the patients' bodies. This could include providing commands to control the location of the stimulator(s) 507 and/or sensor(s) 503 by operating the actuator(s) 505 to adjust the location (e.g., to place a stimulator and/or sensor in contact with skin of a breast, chest, and/or abdomen of a patient, e.g., in contact and pushing into the skin by approximately 0.5 millimeters). Additionally or alternatively, user interface 504 may include elements to allow a patient and/or an experimenter to input whether a patient perceived a provided stimulus and/or to input information related to the patient's perception of the stimulus.

Processor(s) 506 may comprise one or more general purpose processors—e.g., microprocessors—and/or one or more special purpose processors—e.g., digital signal processors (DSPs), graphics processing units (GPUs), floating point units (FPUs), network processors, tensor processing units (TPUs), or application-specific integrated circuits (ASICs). In some instances, special purpose processors may be capable of model execution (e.g., execution of artificial neural networks or other machine learning models) or other functions as described herein, among other applications or functions. Data storage 508 may include one or more volatile and/or non-volatile storage components, such as magnetic, optical, flash, or organic storage, and may be integrated in whole or in part with processor(s) 506. Data storage 508 may include removable and/or non-removable components.

Processor(s) 506 may be capable of executing program instructions 518 (e.g., compiled or non-compiled program logic and/or machine code) stored in data storage 508 to carry out the various functions described herein. Therefore, data storage 508 may include a non-transitory computer-readable medium, having stored thereon program instructions that, upon execution by system 500, cause system 500 to carry out any of the methods, processes, or functions disclosed in this specification and/or the accompanying drawings. The execution of program instructions 518 by processor(s) 506 may result in processor 506 using data 512.

By way of example, program instructions 518 may include an operating system 522 (e.g., an operating system kernel, device driver(s), and/or other modules) and one or more application programs 520 (e.g., functions for executing the methods described herein) installed on system 500. Data 512 may include stored experimental data 516 (e.g., stored sensor inputs, stored inputs indicative of patient percepts, stored quantities derived therefrom related to tactile acuity or other measurements of interest) that can be executed as part of the methods described herein (e.g., to determine, from a set of sensor inputs and corresponding patient percept inputs, a tactile acuity at one or more locations of a patient's breast, chest, or other body part).

Application programs 520 may communicate with operating system 522 through one or more application programming interfaces (APIs). These APIs may facilitate, for instance, application programs 520 transmitting or receiving information via communication interface 502, receiving and/or displaying information on user interface 504, and so on.

Application programs 520 may take the form of “apps” that could be downloadable to system 500 through one or more online application stores or application markets (via, e.g., the communication interface 502). However, application programs can also be installed on system 500 in other ways, such as via a web browser or through a physical interface (e.g., a USB port) of the system 500.

Sensor(s) 503 may include one or more accelerometers, gyroscopes, pressure sensors, displacement sensors, limit switches, temperature sensors, humidity sensors, or other sensors configured to detect one or more physical variable related to stimuli that may be applied to human skin.

Stimulator(s) 507 may include vibrator motors, solenoids, heating elements, electrodes, cooling elements, motors, and/or other elements configured to provide a force, pressure, displacement, electrical voltage or current, temperature or thermal energy flux, or other stimulus to human skin. Sensor(s) 503 may be coupled to the stimulator(s) 507 to facilitate measurement of the relevant physical variable(s). For example, a stimulator could be coupled to a probe via which stimuli are delivered to skin (e.g., the probe is brought into contact with the skin surface, at which point a vibratory stimulus may be delivered to the skin from the stimulator via the probe

Actuator(s) 505 may include motors, servos, pneumatic or hydraulic cylinders, and/or other elements configured to adjust the location of the sensor(s) 503 and/or stimulator(s) 507, thereby placing them into contact with skin of a patient. In some examples, the actuator(s) 505 may be, or may have components in common with, the stimulator(s) 507. For example, if a stimulus to be applied is an indentation force applied to the skin by a probe coupled to the actuator(s) 505, then operation of the actuator to push the probe into the skin (thereby applying the indentation force thereto) also acts to provide the stimulus. In such an example, the actuator(s) 505 act as the stimulator(s) 507.

The system 500 may include an armature (not shown) configured to mechanically couple elements of the system 500 together in order to facilitate the functionality described herein. For example, the sensors 503, actuators 505, and/or stimulators 507 could be coupled to each other and/or to the ground, to a padded platform on which a patient can lay prone such that their breast, chest, and/or abdomen is located within/spans across an aperture of the platform, to a plate which may be in contact with skin of the patient in order to facilitate controlling and/or adjusting the relative location of such elements of the system 500 (e.g., to prevent motion of the patient's breast/chest abdomen related to breathing and also to place a probe or other element configured to provide stimulus thereto in contact with the skin of the patient).

### IV. Example Methods

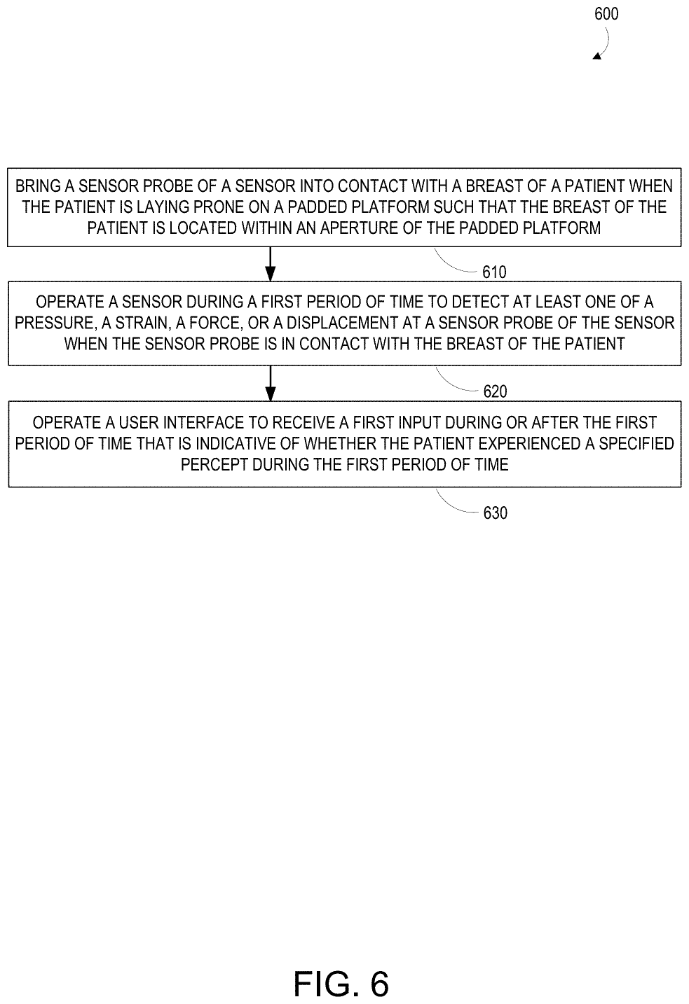

FIG. 6 depicts an example method 600 for quantitatively assessing tactile acuity of a breast of a patient. The method 600 includes bringing a sensor probe of a sensor into contact with a breast of a patient when the patient is laying prone on a padded platform such that the breast of the patient is located within an aperture of the padded platform (610). The method 600 additionally includes operating a sensor during a first period of time to detect at least one of a pressure, a strain, a force, or a displacement at a sensor probe of the sensor when the sensor probe is in contact with the breast of the patient (620). The method 600 also includes operating a user interface to receive a first input during or after the first period of time that is indicative of whether the patient experienced a specified percept during the first period of time (630). The method 600 could include additional steps or features.

It should be understood that arrangements described herein are for purposes of example only. As such, those skilled in the art will appreciate that other arrangements and other elements (e.g. machines, interfaces, operations, orders, and groupings of operations, etc.) can be used instead of or in addition to the illustrated elements or arrangements.

### V. Conclusion

It should be understood that arrangements described herein are for purposes of example only. As such, those skilled in the art will appreciate that other arrangements and other elements (e.g., machines, interfaces, operations, orders, and groupings of operations, etc.) can be used instead, and some elements may be omitted altogether according to the desired results. Further, many of the elements that are described are functional entities that may be implemented as discrete or distributed components or in conjunction with other components, in any suitable combination and location, or other structural elements described as independent structures may be combined.

While various aspects and implementations have been disclosed herein, other aspects and implementations will be apparent to those skilled in the art. The various aspects and implementations disclosed herein are for purposes of illustration and are not intended to be limiting, with the true scope being indicated by the following claims, along with the full scope of equivalents to which such claims are entitled. It is also to be understood that the terminology used herein is for the purpose of describing particular implementations only, and is not intended to be limiting.

We claim:

1. A system, comprising:
a padded platform, wherein the padded platform includes an aperture, and wherein the padded platform is shaped to permit a patient to lay prone on the platform with a breast of the patient located within the aperture;
a sensor configured to detect at least one of a pressure, a force, or a displacement at a sensor probe of the sensor;
an armature, wherein the sensor is coupled to the armature, and wherein the armature is configured to bring the sensor probe into contact with the breast of the patient when the patient is laying prone on the platform such that the breast of the patient is located within the aperture;
a user interface; and
a controller, wherein the controller is operably coupled to the sensor and the user interface, and wherein the controller is configured to:
operate the sensor during a first period of time to detect at least one of a pressure, a strain, a force, or a displacement at the sensor probe when the sensor probe is brought into contact with the breast of the patient; and
operate the user interface to receive a first input during or after the first period of time that is indicative of whether the patient experienced a specified percept during the first period of time.

2. The system of claim 1, wherein the user interface comprises a button that is configured to be pressed by the patient when the patient is laying prone on the platform such that the breast of the patient is located within the aperture.

3. The system of claim 1, further comprising a vibrator coupled to the sensor probe, wherein the controller is additionally configured to, during the first period of time, operate the vibrator to provide a vibratory stimulus.

4. The system of claim 1, wherein the controller is additionally configured to:
operate the sensor during a second period of time to detect at least one of a pressure, a strain, a force, or a displacement at the sensor probe; and
operate the user interface to receive a second input during or after the second period of time that is indicative of whether the patient experienced a specified percept during the second period of time.

5. The system of claim 4, further comprising a stimulator coupled to the armature, wherein the controller is additionally configured to:
operate the stimulator during and/or prior to the first period of time to provide a first stimulus via the sensor probe; and
operate the stimulator during and/or prior to the second period of time to provide a second stimulus via the sensor probe, wherein the first stimulus and second stimulus differ with respect to at least one of pressure, force, displacement, or location at which the first and second stimuli are applied to the breast of the patient.

6. The system of claim 1, wherein the user interface comprises a display, and wherein the controller is additionally configured to:
operate the display of the user interface during or after the second period of time to provide, to a patient laying prone on the platform such that the breast of the patient is located within the aperture, an indication that the patient should provide feedback related to whether the patient experienced the specified percept during the first period of time.

7. The system of claim 1, further comprising a second sensor operably coupled to the controller and the user interface, wherein the second sensor is configured to detect visual changes in the breast when the sensor probe is brought into contact with the breast of the patient.

8. The system of claim 1, further comprising:
a plate, wherein the plate includes a first hole formed therethrough, wherein the plate is coupled to the armature, wherein the armature is configured to bring the plate into contact with the breast of the patient when the patient is laying prone on the platform such that the breast of the patient is located within the aperture, and wherein the armature is configured to bring the sensor probe into contact with the breast of the patient through the first hole of the plate.

9. The system of claim 8, wherein the plate includes a second hole formed therethrough, and wherein the armature is also configured to bring the sensor probe into contact with the breast of the patient through the second hole of the plate.

10. A system, comprising:
a padded platform, wherein the padded platform includes an aperture, and wherein the padded platform is shaped to permit a patient to lay prone on the platform with at least one of: a breast of the patient located within the aperture, a portion of a chest of the patient spanning the aperture, or a portion of an abdomen of the patient spanning the aperture;
a sensor configured to detect at least one of a pressure, a force, or a displacement at a sensor probe of the sensor;
an armature, wherein the sensor is coupled to the armature, and wherein the armature is configured to bring the sensor probe into contact with skin of the breast, chest, or abdomen of the patient when the patient is laying prone on the platform such that at least one of: the breast of the patient is located within the aperture, the portion of the chest of the patient is spanning the aperture, or the portion of the abdomen of the patient is spanning the aperture;
a user interface; and
a controller, wherein the controller is operably coupled to the sensor and the user interface, and wherein the controller is configured to:
operate the sensor during a first period of time to detect at least one of a pressure, a strain, a force, or a displacement at the sensor probe when the sensor probe is brought into contact with the skin of the patient; and
operate the user interface to receive a first input during or after the first period of time that is indicative of whether the patient experienced a specified percept during the first period of time.

11. The system of claim 10, wherein the user interface comprises a button that is configured to be pressed by the patient when the patient is laying prone on the platform such that at least one: of the breast of the patient is located within the aperture, the portion of the chest of the patient is spanning the aperture, or the portion of the abdomen of the patient is spanning the aperture.

12. The system of claim 10, further comprising a vibrator coupled to the sensor probe, wherein the controller is additionally configured to, during the first period of time, operate the vibrator to provide a vibratory stimulus.

13. The system of claim 10, wherein the controller is additionally configured to:
operate the sensor during a second period of time to detect at least one of a pressure, a strain, a force, or a displacement at the sensor probe; and
operate the user interface to receive a second input during or after the second period of time that is indicative of whether the patient experienced a specified percept during the second period of time.

14. The system of claim 13, further comprising a stimulator coupled to the armature, wherein the controller is additionally configured to:
operate the stimulator during and/or prior to the first period of time to provide a first stimulus via the sensor probe; and
operate the stimulator during and/or prior to the second period of time to provide a second stimulus via the sensor probe, wherein the first stimulus and second stimulus differ with respect to at least one of pressure, force, displacement, or location at which the first and second stimuli are applied to the skin of the patient.

15. The system of claim 10, wherein the user interface comprises a display, and wherein the controller is additionally configured to:
operate the display of the user interface during or after the second period of time to provide, to a patient laying prone on the platform, an indication that the patient should provide feedback related to whether the patient experienced the specified percept during the first period of time.

16. The system of claim 10, further comprising a second sensor operably coupled to the controller and the user interface, wherein the second sensor is configured to detect visual changes in the skin when the sensor probe is brought into contact with the skin of the patient.

17. The system of claim 10, further comprising:
a plate, wherein the plate includes a first hole formed therethrough, wherein the plate is coupled to the armature, wherein the armature is configured to bring the plate into contact with the skin of the patient when the patient is laying prone on the platform such that at least one of:
the breast of the patient is located within the aperture, the portion of the chest of the patient is spanning the aperture, or the portion of the abdomen of the patient is spanning the aperture, and wherein the armature is configured to bring the sensor probe into contact with the skin of the patient through the first hole of the plate.

18. The system of claim 17, wherein the plate includes a second hole formed therethrough, and wherein the armature is also configured to bring the sensor probe into contact with the skin of the patient through the second hole of the plate.

19. A method for quantitatively assessing tactile acuity of a breast of a patient, the method comprising:
bringing a sensor probe of a sensor into contact with a breast of a patient when the patient is laying prone on a padded platform such that the breast of the patient is located within an aperture of the padded platform;
operating a sensor during a first period of time to detect at least one of a pressure, a strain, a force, or a displacement at a sensor probe of the sensor when the sensor probe is in contact with the breast of the patient; and
operating a user interface to receive a first input during or after the first period of time that is indicative of whether the patient experienced a specified percept during the first period of time.

20. The method of claim 19, wherein bringing the sensor probe of the sensor into contact with the breast of a patient when the patient is laying prone on the padded platform such that the breast of the patient is located within the aperture of the padded platform comprises bringing the sensor probe of the sensor into contact with the breast of a patient through a hole formed through a plate that is in contact with the breast of the patient when the patient is laying prone on the padded platform.
